(19) United States
(12) Patent Application Publication
(10) Pub. No.: US 2026/0221288 A1
(43) Pub. Date: Jul. 30, 2026

(54) PREDICTING 30 DAY READMISSION FOR ACUTE HEART FAILURE USING MACHINE LEARNING

(71) Applicant: REGENTS OF THE UNIVERSITY OF MICHIGAN, Ann Arbor, MI (US)

(72) Inventors: Brittany Baur, Ann Arbor, MI (US); Sardar Ansari, Ypsilanti, MI (US); Brandon C. Cummings, White Lake, MI (US); Kevin R. Ward, Superior Township, MI (US); Taylor Lebeis, Ann Arbor, MI (US); Kyle Gunnerson, Saline, MI (US); Connor O'Brien, Ann Arbor, MI (US); Joseph Blackmer, Ann Arbor, MI (US)

(21) Appl. No.: 19/458,413

(22) Filed: Jan. 23, 2026

Related U.S. Application Data
(60) Provisional application No. 63/749,156, filed on Jan. 24, 2025.

(51) Int. Cl. G16H 50/30; G16H 10/60; G16H 50/20

(52) U.S. Cl. CPC G16H 50/30; G16H 50/20; G16H 10/60

(57) ABSTRACT

Systems and methods for predicting readmission of a patient (e.g., within 30 days) for acute heart failure using machine learning are provided. The computer-implemented method includes receiving an electronic health record (EHR) dataset of the patient proximate a discharge event associated with the patient and a care facility, wherein the EHR dataset indicates the patient, while admitted to the care facility, is diagnosed with acute heart failure and receives a medication associated with acute heart failure. The method includes determining a risk metric corresponding to a predicted risk of readmission of the patient for acute heart failure by analyzing the EHR dataset of the patient using a trained machine learning model, and when the risk metric exceeds a threshold metric, outputting an output associated with the risk metric at an output device.

### CROSS-REFERENCE TO RELATED APPLICATIONS

This application claims the priority benefit under 35 U.S.C. § 119(e) of U.S. Provisional Application No. 63/749,156, filed Jan. 24, 2025, which is incorporated herein by reference in its entirety.

### FIELD OF THE DISCLOSURE

The present disclosure generally relates to predicting readmission of a patient to a care facility, and more particularly, to systems and methods for using machine learning to predict readmission of a patient (e.g., within 30 days) for acute heart failure.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Insurers can penalize care facilities, such as hospitals, when a patient is readmitted back into the care facility within 30 days of discharge for the same or a related illness, for example by reducing payments to the care facility that has excess readmissions that may indicate overlooked complications or improper patient treatment at the care facility. Patients admitted into the care facility to treat acute heart failure (AHF) in particular have high rates of readmission. Thus, predicting a patient's risk of readmission for AHF or other diseases can be an important part of clinical practice, as identifying patients at high risk for readmission allows the care facility to design early intervention strategies to slow disease progression, delay patient discharge, and/or otherwise avoid insurer penalization due to patient readmission.

Conventional techniques may predict a patient's risk for developing a disease using their electronic health record (EHR) information, however, such predictions generally do not include the risk of readmission of a patient for the same/similar disease as that which they were previously admitted, and do not focus on analyzing recent EHR information gathered while the patient is admitted that may be most indicative of their risk of readmittance after discharge. Thus, a need exists for improved techniques to predict a patient's risk of readmission for a disease, particularly acute heart failure, within 30 days from the time of discharge to address the aforementioned inefficiencies, shortcomings, and/or other inadequacies.

### BRIEF SUMMARY

In one aspect, a method for predicting readmission of a patient for acute heart failure using machine learning includes: receiving, via one or more processors, an electronic health record (EHR) dataset of the patient proximate a discharge event associated with the patient and a care facility, wherein: the EHR dataset indicates the patient, while admitted to the care facility, is diagnosed with acute heart failure and receives a medication associated with acute heart failure; determining, via the one or more processors, a risk metric corresponding to a predicted risk of readmission of the patient for acute heart failure by analyzing the EHR dataset of the patient using a trained machine learning model; and when the risk metric exceeds a threshold metric, outputting, via one or more processors, an output associated with the risk metric at an output device.

In another aspect, a system for predicting readmission of a patient for acute heart failure using machine learning includes one or more processors, and a memory including computer executable instructions that, when executed by the one or more processors, cause the system to: receive an electronic health record (EHR) dataset of the patient proximate a discharge event associated with the patient and a care facility, wherein: the EHR dataset indicates the patient, while admitted to the care facility, is diagnosed with acute heart failure and receives a medication associated with acute heart failure; determine a risk metric corresponding to a predicted risk of readmission of the patient for acute heart failure by analyzing the EHR dataset of the patient using a trained machine learning model; and when the risk metric exceeds a threshold metric, output an output associated with the risk metric at an output device.

In yet another aspect, a non-transitory computer readable medium storing instructions that, when executed by one or more processors, causes the one or more processors to at least receive an electronic health record (EHR) dataset of a patient proximate a discharge event associated with the patient and a care facility, wherein: the EHR dataset indicates the patient, while admitted to the care facility, is diagnosed with acute heart failure and receives a medication associated with acute heart failure; determine a risk metric corresponding to a predicted risk of readmission of the patient for acute heart failure by analyzing the EHR dataset of the patient using a trained machine learning model; and when the risk metric exceeds a threshold metric, output an output associated with the risk metric at an output device.

### BRIEF DESCRIPTION OF THE DRAWINGS

The figures described below depict various aspects of the applications, methods, and systems disclosed herein. It should be understood that each figure depicts one embodiment of a particular aspect of the disclosed applications, systems and methods, and that each of the figures is intended to accord with a possible embodiment thereof. Furthermore, wherever possible, the following description refers to the reference numerals included in the following figures, in which features depicted in multiple figures are designated with consistent reference numerals.

FIG. 1 depicts a block diagram of an example computing environment for implementing systems and methods for predicting readmission of a patient for acute heart failure using machine learning, according to embodiments.

FIG. 2A depicts a combined block and logic diagram for example training of a machine learning model, according to some embodiments.

FIG. 2B depicts a block diagram of an example neural network trained to predict patient readmission and/or mortality from AHF, according to embodiments.

FIG. 2C depicts example metrics associated with evaluating model training, according to embodiments.

FIG. 2D depicts an example table indicating a performance metric for evaluating similar predictive models, according to embodiments.

FIG. 3 depicts an example clinical workflow for predicting readmission of a patient within 30 days for acute heart failure using machine learning, according to embodiments.

FIG. 4 depicts a flow diagram of an example computer-implemented method 400 for predicting readmission of a patient for acute heart failure using machine learning, according to embodiments.

Advantages will become more apparent to those skilled in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### DETAILED DESCRIPTION

### Overview

The present disclosure describes systems and techniques that overcome limitations of conventional systems. The disclosed techniques include methods and systems for predicting readmission of a patient (e.g., within 30 days) for acute heart failure using machine learning. As used herein, a “patient” may include any person receiving treatment or otherwise care for acute heart failure (AHF) at a care facility (e.g., clinic, hospital). The present techniques include using machine learning to analyze a patient's electronic health record (EHR) dataset at the time of patient discharge, where the patient is diagnosed with AHF and receives a medication associated with acute heart failure (e.g., intravenous diuretics) while admitted. The machine learning model may determine a risk metric (e.g., score) corresponding to a predicted risk of readmission of the patient within 30 days for acute heart failure based upon analyzing the EHR dataset of the patient, such as analyzing the EHR data generated during the patient admittance for AHF. In at least some embodiments, at least one monitoring device may gather cardiovascular data from the patient while admitted and provide the cardiovascular data to the machine learning model (e.g., via the EHR dataset) as an additional input which may improve the accuracy of the risk metric. Such example monitoring devices may include, but are not limited to, ECG/EKG devices, blood pressure devices, devices measuring cardiac output, central venous pressure, lung water, respiratory pressure, respiratory volume, respiratory flow, respiration rate, work of breathing, heart rate variability, arterial vascular waveforms, etc. When the risk metric exceeds a threshold metric (e.g., indicating a risk of patient readmittance after discharge for AHF), an information associated with the risk metric may be output at an output device, such as displaying the risk metric or associated risk classification (e.g., low, medium, or high risk) at a computing device of the caregiver for determine the best course of patient treatment proximate the time of discharge to avoid readmittance for acute heart failure.

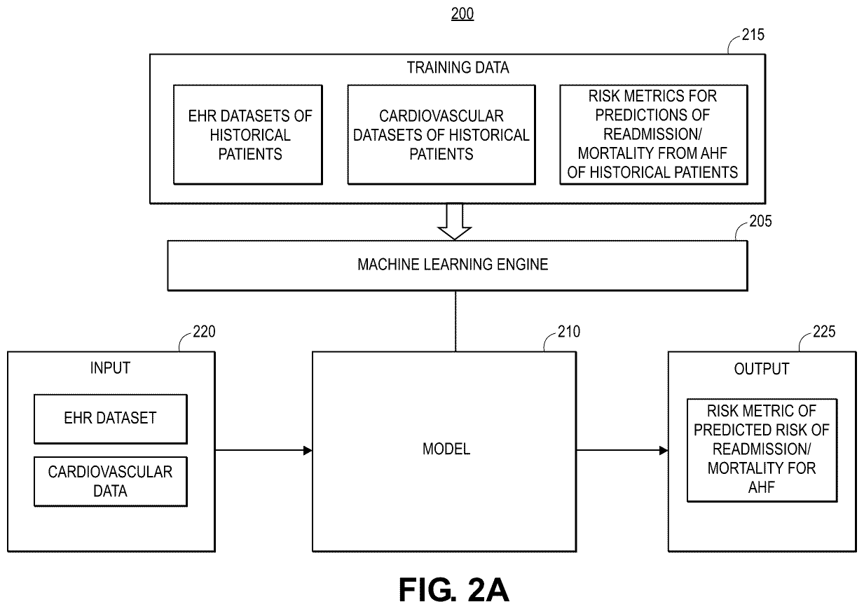

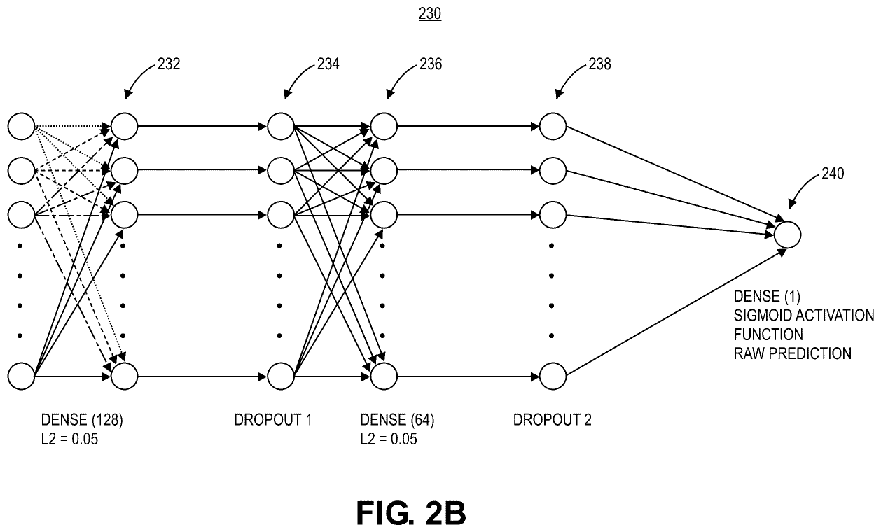

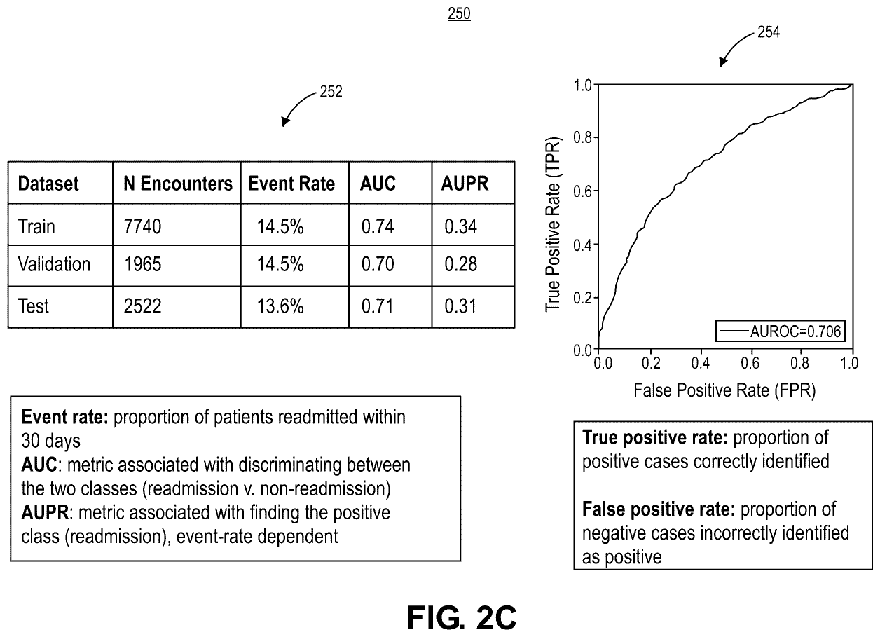

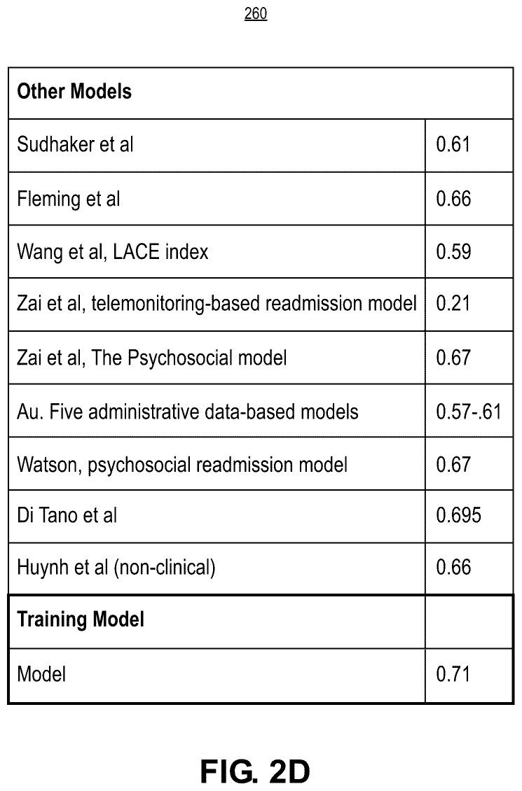

The present techniques use a state-of-the-art machine learning architecture (e.g., neural network) that can be seamlessly integrated into an EHR system to support a clinical workflow for AHF patient treatment. In general, the present systems and methods employ more accurate and explicable techniques than those known in the art to predict patient readmittance after discharge, namely for AHF, as further described herein with respect to FIG. 2D. For example, the machine learning architecture of the present techniques may have an area under the receiver operating characteristic curve (AUROC) value of 0.71, outperforming other models with lower AUROC values, and may have an area under the precision-recall curve (AUPR or AUPRC) of 0.31, indicating good predictive performance. The disclosed systems and methods may output information associated with the risk metric indicating or otherwise explaining the predicted patient readmission risk and/or other associated information (e.g., Shapley features of the patient EHR data) influencing the predictive risk metric, without requiring extra work on the part of the caregiver, and/or changes to the care environment (e.g., the hospital). The result is machine learning-based clinical support tool configured to identify patients at high-risk for readmission, or in some instances death, due to AHF, by analyzing recent EHR information from their admittance for AHF. The machine learning architecture includes the improved ability to process unstructured and structured EHR data and/or cardiovascular data (e.g., of a third party or monitoring device) respective to conventional models only able to analyze structured EHR data, may include dropout layers and L2 regularization that beneficially reduce model overfitting, may include early stopping to reduce overfitting and improve generalization, and may predict a risk of readmission for dozens, hundreds, or thousands of patients.

The present techniques herein, through the machine learning architectures described, is directed to technical improvements in computer-implemented systems for processing heterogeneous medical data and operating in clinical environments wherein readmission analysis is applied. In contrast to conventional computing systems, the present techniques provide reliable processing of multimodal, asynchronous, and noisy clinical data streams such as longitudinal electronic health records (EHRs) in combination with real-time or near real-time physiological data generated by distributed monitoring devices. In various examples, machine learning architectures may be trained by transforming heterogeneous medical data sources into a unified computational representation suitable for machine learning. Electronic health record data, physiological sensor streams, clinical notes, and demographic variables are inherently heterogeneous in structure, temporal resolution, and semantic content. The machine learning architectures herein may be trained through temporal normalization of such heterogenous data, in particular for readmission risk assessment, conversion of unstructured data into structured feature representations using medically constrained tokenization and embedding techniques, in particular regarding readmission risk assessment. In various examples, semantic normalization may be formed on the heterogenous data, as well as synchronization to preserve temporal relationships. In various examples herein, machine learning architectures are able to perform such operations during training and during inference operation of a trained machine learning model, thereby providing faster more efficient risk assessment of readmission risk and to prove such technical improvements to distributed medical systems. For example, trained machine learning models from the machine learning architectures herein may be deployed at point of care computing systems advising attending care providers for readmission risk for acute heart failure prior to patient discharge. The machine learning architectures herein may thus be deployed, in some examples, in closed-loop adaptive systems that can (in addition to sending risk assessments to care providers) be used to modify heterogeneous data collection streams to prioritize data collection for data most relevant to readmission risk assessment, to allow for real-time detection of changes in readmission risk assessment, to adjust monitoring device operation based on readmission risk assessment, and to dynamically adjust which heterogenous data is relevant to the same and to adjust temporal relationships between such data, as a subjects condition changes.

### Example Computing Environment

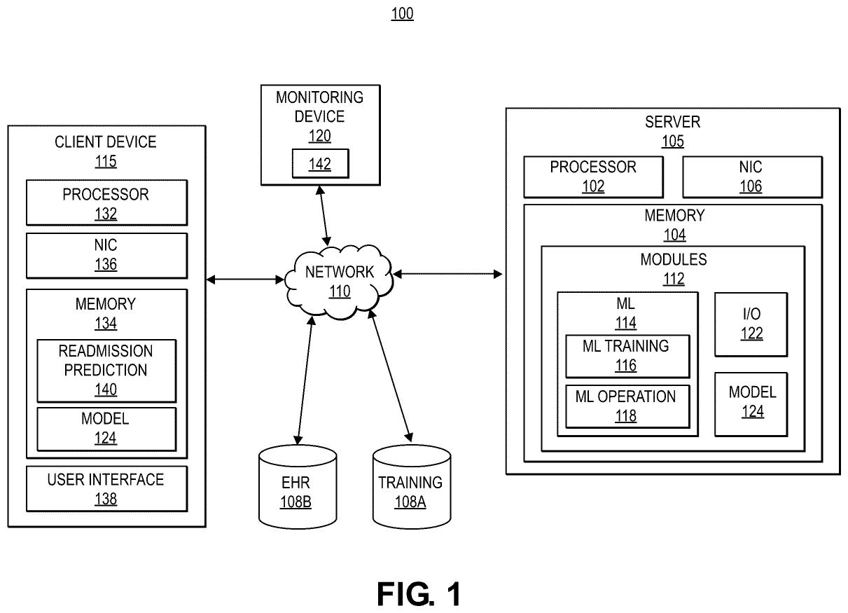

FIG. 1 depicts a block diagram of an example computing environment 100 for implementing systems and methods for predicting readmission of a patient for acute heart failure using machine learning, according to embodiments. The computing environment 100 may include a server 105 communicatively coupled, via a network 110, to one or more databases 108, a client device 115, and/or a monitoring device 120. In at least some embodiments, the computing environment 100 may comprise a cloud-based electronic health record (EHR) system. For example, the client device 115 may be located at a care facility such as a hospital, the server 105 may be a cloud server processing, among other things, EHR data, and the monitoring device 120 may monitor patient health at the care facility, all of which communicate and/or otherwise share data via the network 110. Although FIG. 1 depicts certain entities, components, equipment, and devices, it should be appreciated that additional or alternate entities, components, equipment, and devices are envisioned.

The server 105 may perform one or more functionalities associated with predicting a risk of readmission of a patient within a time frame such as 30 days, 60 days, etc. for acute heart failure, such as obtaining patient EHR datasets, training and/or operating a machine learning model that determines a risk metric predicting the risk of readmission for AHF, etc. The server 105 may be part of a cloud network or may otherwise communicate with other hardware or software components within one or more cloud computing environments to send, retrieve, or otherwise analyze data or information described herein. For example, in certain aspects of the present techniques, the computing environment 100 may comprise an on-premise computing environment, a multi-cloud computing environment, a public cloud computing environment, a private cloud computing environment, and/or a hybrid cloud computing environment. For example, an entity (e.g., a medical institution) may host one or more services in a public cloud computing environment (e.g., Alibaba Cloud, Amazon Web Services (AWS), Google Cloud, IBM Cloud, Microsoft Azure, etc.). The public cloud computing environment may be a traditional off-premise cloud (i.e., not physically hosted at a location owned/controlled by the medical institution). Alternatively, or in addition, aspects of the public cloud may be hosted on-premises at a location owned/controlled by the medical institution. The public cloud may be partitioned using visualization and multi-tenancy techniques and may include one or more infrastructure-as-a-service (IaaS) and/or platform-as-a-service (PaaS) services.

The server 105 may include at least one processor 102. The processor 102 may include one or more suitable processors (e.g., central processing units (CPUs) and/or graphics processing units (GPUs)). The processor 102 may be connected to a memory 104 via a computer bus (not depicted) responsible for transmitting electronic data, data packets, or otherwise electronic signals to and from the processor 102 and memory 104 in order to implement or perform the machine-readable instructions, methods, processes, elements, or limitations, as illustrated, depicted, or described for the various flowcharts, illustrations, diagrams, figures, and/or other disclosure herein. The processor 102 may interface with the memory 104 via a computer bus to execute an operating system (OS) and/or computing instructions contained therein, and/or to access other services. For example, the processor 102 may interface with the memory 104 via the computer bus to create, read, update, delete, or otherwise access or interact with the data stored in the memory 104.

The memory 104 may include one or more forms of volatile and/or non-volatile, fixed and/or removable memory, such as read-only memory (ROM), electronic programmable read-only memory (EPROM), random access memory (RAM), erasable electronic programmable read-only memory (EEPROM), and/or other hard drives, flash memory, MicroSD cards, and others. The memory 104 may store an operating system (OS) (e.g., Microsoft Windows, Linux, UNIX, etc.) capable of facilitating the functionalities, apps, methods, or other software as discussed herein.

In general, a computer program or computer based product, application, or code (e.g., the model(s), such as ML models, or other computing instructions described herein) may be stored on a computer usable storage medium, or tangible, non-transitory computer-readable medium (e.g., standard random access memory (RAM), an optical disc, a universal serial bus (USB) drive, or the like) having such computer-readable program code or computer instructions embodied therein, wherein the computer-readable program code or computer instructions may be installed on or otherwise adapted to be executed by the processor(s) 102 (e.g., working in connection with the respective operating system in memory 104) to facilitate, implement, or perform the machine readable instructions, methods, processes, elements or limitations, as illustrated, depicted, or described for the various flowcharts, illustrations, diagrams, figures, and/or other disclosure herein. In this regard, the program code may be implemented in any desired program language, and may be implemented as machine code, assembly code, byte code, interpretable source code or the like (e.g., via Golang, Python, C, C++, C#, Objective-C, Java, Scala, ActionScript, JavaScript, HTML, CSS, XML, etc.).

The server 105 may include, and/or have access to (e.g., via the network 110) at least one database 108. The database 108 may be a relational database, such as Oracle, DB2, MySQL, a NoSQL based database, such as MongoDB, or another suitable database. The database 108 may store datasets including one or more types of data, records, files, etc., and/or other data, however, the term data and dataset may be used interchangeably herein. Some types of data and/or datasets stored in the database 108, memory 104, and/or otherwise available to the computing environment 100 may be private datasets (e.g., medical information of patients), or any other suitable type of data.

The computing environment 100 may include a model training database 108A storing datasets to train and/or operate one or more models, as further described below with respect to FIG. 2A. The model training database 108A may store electronic health record dataset of a plurality of historical patients, cardiovascular data of a plurality of historical patients, predictions of readmissions due to AHF of a plurality of historical patients, and/or any other suitable training data for a model 124.

The computing environment 100 may include an EHR database 108B storing electronic health record (EHR) datasets of patients, also referred to as electronic medical record (EMR) datasets. The EHR database 108B may store various types of EHR data associated with patients, which may include features indicating the severity of the patient's AHF and/or their risk of readmission or mortality from AHF. The EHR datasets may include data (e.g., structured data, unstructured data, real-time data) provided by the one or more devices, such as devices associated with monitoring the patient during the admission, such as the monitoring device 120, an electrocardiogram device, and/or other suitable patient monitoring devices. The EHR dataset may include or otherwise indicate, but is not limited to, vital signs (e.g., blood pressure, Glascow Coma Scale (GCS), height, pulse, respiratory rate, supplemental oxygen, oxygen saturation, temperature, urine output, weight); lab results (e.g., INR, PTT, PT (in seconds), albumin, bicarbonate, bun, calcium, chloride, CO2, creatinine, glucose, hematocrit, hemoglobin, lactate, magnesium, mean corpuscular hemoglobin, Mean corpuscular hemoglobin concentration, mean corpuscular hemoglobin volume, mean platelet volume, phosphorus, platelets, potassium, total protein, red blood cell count, red cell distribution width, sodium, white blood cell count, bilirubin, pH, lactate, partial pressure oxygen, partial pressure CO2, troponin, brain natriuretic peptide, N-terminal prohormone of brain natriuretic peptide); demographics (e.g., gender, race, age); chief complaint (e.g., reason for visit); comorbidities (e.g., indicated by the Elixhauser comorbidity index); encounter diagnosis (e.g., additional diagnoses aside from AHF); selected treatments or procedures (e.g., life vest, pacemaker, other cardiac-related treatments), heart catheterization (e.g., left and right), life vest, ablation, cardioversion, pacemaker, continuous resynchronization therapy (CRT), continuous renal replacement therapy (CRRT), hemodialysis, paracentesis, thoracentesis, balloon pump, impella, tandom heart ECMO, inotropy); Medications (e.g., Inotropes (milrinone, dobutamine), ACE (angiotensin-coverting enzyme) inhibitors, ARB (angiotensin receptor blocker), ARNI (angiotension receptor/neprilysin inhibitor), beta blockers, long-acting nitrates, aldosterone antagonists, SGLT2 (sodium-glucose cotransporter-2) inhibitors, hydralazine, loop diuretics (lasix, torsemide, bumex), thiazide diuretics (hydrochlorothiazide, chlorothalidone, metolazone), anti-arrythmics (amiodarone), vericiguat); length to previous encounter; discharge disposition (e.g., discharge order); a current hospital unit the patient is admitted to; level of care patient's (e.g., level of care at discharge), and/or other suitable EHR data of the patient.

The EHR datasets may include cardiovascular data of the patient, such as cardiovascular data provided by at least one monitoring device 120. The cardiovascular data may include, and/or indicate, arterial and/or venous waveforms and/or pressures, cardiac output/index, lung water, respiratory waveforms and/or pressures, exhaled breath markers of inflammation, exhaled water concentrations, derived values such as shock index, systemic or peripheral vascular resistance, and/or any other suitable cardiovascular data of the patient. The cardiovascular data, which may be generally associated with the heart, blood vessels, and or blood circulation of the patient, may also include cardiopulmonary data (e.g., generated or otherwise provided by the provided by one or more monitoring devices 120). The cardiopulmonary data may include, or otherwise indicate, information associated with the heart and lungs (e.g., oxygen and carbon dioxide exchange) of the patient. Accordingly, the cardiopulmonary data may further include and/or indicate metrics, values, waveforms, signals, pressures, indexes, and the like, associated with spirometry, blood oxygen saturation (SpO2), arterial blood gas, peak expiratory flow, respiratory rate, diffusion capacity, ventilation and/or perfusion, oxygen uptake efficiency slopes, and the like.

The memory 104 may store a plurality of computing modules 112, implemented as respective sets of computer-executable instructions (e.g., one or more source code libraries) as described herein. The computing modules 112 may include an ML module 114. The ML module 114 may include ML training module (MLTM) 116 and/or ML operation module (MLOM) 118. In some embodiments, at least one of a plurality of ML methods and algorithms may be applied by the ML module 114, which may include, but are not limited to: linear or logistic regression, instance-based algorithms, regularization algorithms, decision trees, Bayesian networks, cluster analysis, association rule learning, artificial neural networks, deep learning, combined learning, reinforced learning, dimensionality reduction, and support vector machines. In various embodiments, the implemented ML methods and algorithms are directed toward at least one of a plurality of categorizations of ML, such as supervised learning, unsupervised learning, and reinforcement learning. In one aspect, the ML based algorithms may be included as a library or package executed on server(s) 105. For example, libraries may include the TensorFlow based library, the PyTorch library, and/or the scikit-learn Python library.

In one embodiment, the ML module 114 employs supervised learning, which involves identifying patterns in existing data to make predictions about subsequently received data. Specifically, the ML module is “trained” (e.g., via the MLTM 116) using training data, which includes exemplary inputs and associated exemplary outputs. Based upon the training data, the ML module 114 may generate a predictive function which maps outputs to inputs and may utilize the predictive function to generate ML outputs based upon data inputs. The exemplary inputs and exemplary outputs of the training data may include any of the data inputs or ML outputs described above. In the exemplary embodiments, a processing element may be trained by providing it with a large sample of data with known characteristics or features.

In another embodiment, the ML module 114 may employ unsupervised learning, which involves finding meaningful relationships in unorganized data. Unlike supervised learning, unsupervised learning does not involve user-initiated training based upon exemplary inputs with associated outputs. Rather, in unsupervised learning, the ML module 114 may organize unlabeled data according to a relationship determined by at least one ML method/algorithm employed by the ML module 114. Unorganized and/or unstructured data may include any combination of data inputs and/or ML outputs as described above.

In yet another embodiment, the ML module 114 may employ reinforcement learning, which involves optimizing outputs based upon feedback from a reward signal. Specifically, the ML module 114 may receive a user-defined reward signal definition, receive a data input, utilize a decision-making model to generate the ML output based upon the data input, receive a reward signal based upon the reward signal definition and the ML output, and alter the decision-making model so as to receive a stronger reward signal for subsequently generated ML outputs. Other types of ML may also be employed, including deep or combined learning techniques.

The MLTM 116 may comprise a set of computer-executable instructions implementing ML training (e.g., model creation, fine-tuning, retraining, etc.). The MLTM 116 may access one or more databases 108 (e.g., the model training database 108A) or any other data source for training data suitable to generate and/or otherwise train one or more ML models. The training data may be sample data with assigned relevant and comprehensive labels (classes or tags) used to fit the parameters (weights) of an ML model with the goal of training it by example. In one aspect, once an appropriate ML model is trained and validated to provide accurate predictions and/or responses, the trained model may be loaded into MLOM 118 at runtime to process input data and generate output data.

The MLTM 116 may receive labeled data at an input layer of a model having a networked layer architecture (e.g., an artificial neural network, a convolutional neural network, etc.) for training the one or more ML models. The received data may be propagated through one or more connected deep layers of the ML model to establish weights of one or more nodes, or neurons, of the respective layers. Initially, the weights may be initialized to random values, and one or more suitable activation functions may be chosen for the training process. The present techniques may include training a respective output layer of the one or more ML models. The output layer may be trained to output a prediction, for example.

The MLOM 118 may comprise a set of computer-executable instructions implementing ML loading, configuration, initialization and/or operation functionality. The MLOM 118 may include instructions for storing trained models (e.g., in the electronic database 108). As discussed, once trained, the one or more trained ML models may be operated in inference mode, whereupon when provided with de novo input that the model has not previously been provided, the model may output one or more predictions, classifications, etc., as described herein.

While various embodiments, examples, and/or aspects disclosed herein may include training and generating one or more ML models for the server 105 to load at runtime, it is also contemplated that one or more appropriately trained ML models may already exist (e.g., in database 108) such that the server 105 may load an existing trained ML model at runtime. It is further contemplated that the server 105 may retrain, fine-tune, update and/or otherwise alter an existing ML model before and/or after loading the model at runtime.

In one aspect, the computing modules 112 may include an input/output (I/O) module 122, comprising a set of computer-executable instructions implementing communication functions. The I/O module 122 may include a communication component configured to communicate (e.g., send and receive) data via one or more external/network port(s) to one or more networks or local terminals, such as the computer network 110 and/or the client device 115 (for rendering or visualizing) described herein. In one aspect, the servers 105 may include a client-server platform technology such as ASP.NET, Java J2EE, Ruby on Rails, Node.js, a web service or online application programming interface (API), responsive for receiving and responding to electronic requests. I/O module 122 may further include or implement an operator interface configured to present information to an administrator or operator and/or receive inputs from the administrator and/or operator. An operator interface may provide a display screen. The I/O module 122 may facilitate I/O components (e.g., ports, capacitive or resistive touch sensitive input panels, keys, buttons, lights, LEDs), which may be directly accessible via, or attached to, servers 105 or may be indirectly accessible via or attached to the client device 115. According to one aspect, an administrator or operator may access the servers 105 via the client device 115 to review information, make changes, input training data, initiate training via the MLTM 116, and/or perform other functions (e.g., operation of one or more trained models via the MLOM 118).

The computing modules 112 may include the machine learning model 124 trained to determine a risk metric corresponding a predicted risk of readmission of the patient (e.g., within 30 days, within 60 days, etc.) for AHF, also referred to as chronic heart failure (e.g., due to multiple patient admissions for AHF). The machine learning model 124 may obtain, receive, (e.g., from the monitoring device 120, one or more databases 108) and/or otherwise analyze the EHR dataset and/or cardiovascular data of the patient. In at least some embodiments, the machine learning model 124 is trained to predict the risk of mortality of the patient (e.g., via an associated risk metric) from AHF within 30 days of discharge and/or predict the risk readmission of the patient within 60 days (e.g., via an associated risk metric) from discharge for AHF. The machine learning model 124 may generate one or more risk scores, predictions, and/or other outputs based upon receiving patient EHR data collected while the patient is admitted for AHF. In at least some embodiments, the machine learning model 124 may generate the output further based upon monitoring data (e.g., patient vitals, cardiovascular data, etc.) captured by the monitoring device 120 while the patient is admitted for AHF. The machine learning model 124 may analyze different features (e.g., labs, vitals, comorbidities, medications, etc., such as the EHR data described above respective to the EHR database 108B) and/or feature values (e.g., single value of a feature, the minimum and/or maximum value of a features within 48 hours prior to discharge, etc.) of the input data to generate a respective output of the various outputs. For example, features such as heart rate, respiratory rate, blood pressure, and prior diagnoses of the patient may be of greater important, or more indicative, than other features of one or more a risk of readmission within 30-days of discharge or a risk of mortality within 30 days of discharge.

In at least some embodiments, the machine learning model 124 may include a neural network. In some such embodiments, the neural network may be trained using a binary cross-entropy loss function and/or include a first dense layer, a first dropout layer, a second dense layer, a second dropout layer, and a third dense layer. In some such embodiments, the first dense layer may include L2 regularization, the second dense layer may include L2 regularization, and/or the third dense layer may include a single neuron having a sigmoid activation function. The machine learning model 124 may be trained using training data (e.g., stored in the model training database 108A) that includes historical EHR datasets of a plurality of patients, historical cardiovascular data of a plurality of patients, historical predictions of readmission from AHF within 30 days, readmission from AHF within 60 days, readmission from AHF within other time frames (e.g., within 15 days, within 45 days, etc.) and/or death from AHF, and/or any other suitable training data, as further described herein.

The network 110 may comprise any suitable network or networks, including a local area network (LAN), wide area network (WAN), Internet, or combination thereof. For example, the network 110 may include a wireless cellular service (e.g., 4G, 5G, 6G, etc.). Generally, the network 110 enables bidirectional communication between the servers 105 and a client device 115. In one aspect, the network 110 may comprise a cellular base station, such as cell tower(s), communicating to the one or more components of the computing environment 100 via wired/wireless communications based upon any one or more of various mobile phone standards, including NMT, GSM, CDMA, UMTS, LTE, 5G, 6G, and/or the like. Additionally, or alternatively, the network 110 may comprise one or more routers, wireless switches, or other such wireless connection points communicating to the components of the computing environment 100 via wireless communications based upon any one or more of various wireless standards, including by non-limiting example, IEEE 402.11a/ac/ax/b/c/g/n (Wi-Fi), Bluetooth, and/or the like.

The computing environment 100 may include at least one client device 115. The client device 115 may comprise one or more computers, such as multiple, redundant, or replicated client computers accessed by one or more users. The client device 115 may access devices, services, and/or other components of the computing environment 100 via the network 110, such as accessing the EHR database 108B to obtain a patient EHR dataset, accessing the server 105 to generate a prediction of readmission, etc. The client device 115 may include a desktop computer, laptop, mobile computing device, augmented, virtual, mixed, and/or extended reality glasses/headsets, chatbots, and/or other electronic or electrical components. The client device 115 may include a processor 132 (e.g., the processor 102), a memory 134 (e.g., the memory 104), and a NIC 136 (e.g., the NIC 106). The client device 115 may include at least one user interface 138, such as one or more components to receive an input and/or generate an output. The user interface 138 may include one or more of a keyboard, a mouse, a touchscreen, a microphone, a speaker, an imaging device, a display, etc.

The client device 115 may store a readmission prediction module 140 in the memory 134. The readmission prediction module 140 may be, or include, a program, application, plug-in (e.g., to access EHR system resources like Epic™ or University of Michigan MiChart™ systems), etc. The readmission prediction module 140 may generate (e.g., at the client device 115) and/or otherwise receive (e.g., from the server 105) the prediction of risk of readmission of a patient for AHF (e.g., readmission within 30 days of discharge), and/or other output/prediction of the machine learning model 124. In one example, the readmission prediction module 140 may generate a request for the readmission prediction at client device 115, transmit the request to the server 105 via the network 110, and in response cause the server 105 to generate and transmit the readmission prediction to the client device 115. In other embodiments, the readmission prediction module 140 may generate the readmission prediction at the client device 115, e.g., by obtaining the EHR dataset of a patient (e.g., from the EHR database 108B via the network 110), executing the machine learning model 124 (e.g., stored in the memory 134, obtained from the server 105, etc.), and providing a patient dataset to the machine learning model 124 as an input to generate the readmission prediction. It at least some embodiments, the readmission prediction module 140 may store the predictions in the EHR of the patient, display the predicted risk of readmission at a user interface 138 (e.g., a display of the patient caregiver), and/or transmit the predicted risk of readmission to another device (e.g., the server 105 and/or model training database 108A for updating the machine learning model 124).

The computing environment 100 may include at least one monitoring device 120. The monitoring device 120 may generate cardiovascular data of the patient via monitoring vitals or other biological conditions of the patient via one or more sensors 142 and/or at one or more times (e.g., during admittance of the patient to the healthcare facility). The one or more sensors 142 may measure, and/or the cardiovascular data may indicate, one or more of temperature, blood pressure, pulse, blood oxygen (SpO2) levels, lung airflow, or other suitable vitals and/or biological conditions of the patient as previously described. The monitoring device 120 may include a wearable device (e.g., portable heart monitor, smart watch, smart ring, etc.) and/or other suitable monitoring devices (e.g., spirometer, electrocardiograph, etc.). In at least some embodiments, the monitoring device 120 includes one or more sensors 142 (e.g., impedance sensors) to obtain cardiovascular data of the patient. The monitoring device 120 may store the cardiovascular data from monitoring the patient locally in a memory of the monitoring device 120, store the cardiovascular data remotely (e.g., in the patient's EHR in the EHR database 108B), transmit the cardiovascular data (e.g., via the network 110, API calls, etc.) to another device or component of the computing environment 100 (e.g., to the client device 115, the readmission prediction module 140, the server 105, the machine learning model 124), etc., at one or more times. In at least some embodiments, the machine learning model 124 receives the cardiovascular data collected by the monitoring device 120 as an input to generate the predicted risk of readmission. For the purposes of discussion herein, the EHR of the patient will generally include the cardiovascular data the monitoring device 120 collects from the patient. The cardiovascular data the monitoring device 120 collects, such as cardiovascular data, may increase the accuracy of the risk metric or otherwise prediction the machine learning model 124 generates, for example, by providing cardiovascular data otherwise unavailable in the EHR of the patient, providing real-time cardiovascular data proximate discharge of the patient, etc.

In operation, a patient admitted for, or otherwise diagnosed with, AHF at the healthcare facility may be ready for discharge. The patient discharge may be associated with a discharge event, such as an electronic discharge request, the addition of information into the patient EHR information indicating an impending or recommended discharge, and/or other suitable discharge event. A client device 115 at the care facility may generate (e.g., via the readmission prediction module 140) or otherwise obtain (e.g., from the server 105) a predicted risk of readmission for the patient based upon the patient's EHR at, nor, or otherwise proximate the time of patent discharge to reduce the risk of readmission of the patient within 30 days for AHF. For example, the client device 115 at the care facility may include the readmission prediction module 140 that is integrated with a medical records platform that stores patient EHR datasets in the EHR database 108B. The care provider may generate an electronic discharge order associated with the patient that is stored in the patient's EHR and causes the readmission prediction module 140 to obtain the predicted risk of readmission, although generating the predicted risk of readmission may be based upon other triggers, a manual request of a user of the client device 115 (e.g., via the readmission prediction module 140), and/or in any suitable manner. For example, the readmission prediction module 140 may monitor patient EHR dataset to determine (e.g., based upon ICD codes in the patient EHR) that a patient is admitted to the care facility for acute heart failure.

In at least some embodiments, the readmission prediction module 140 may determine criteria associated with the patient. The criteria may be used to determine whether to generate a predicted risk of readmission for the patient. In one example, the readmission prediction module 140 may determine whether the patient receives intravenous diuretics (e.g., furosemide, bumetanide) or other medication(s) associated with AHF such as nitroglycerin, while admitted to the care facility with acute heart failure. Accordingly, it should be understood while intravenous diuretics are generally described herein, the prediction module 140 may be trained or otherwise configured to receive inputs, analyze data, and/or make predictions associated with other types of medication associated with AHF. For example, the readmission prediction module 140 may analyze ICD codes in the patient's EHR to determine whether diuretics like furosemide or bumetanide were administered intravenously to the patient as an indicator the patient experiences AHF during admittance to the healthcare facility. Although for purposes of discussion The readmission prediction module 140 may determine other criteria associated with the patient receiving the predicted risk of readmission, such as whether the intravenous diuretics are administered to the patient while in the ICU or admitted on a general floor (e.g., as opposed to the emergency room).

Requesting the predicted risk of readmission may cause the server 105 to retrieve the patient's EHR dataset from the EHR database 108B, and/or receive cardiovascular data from the monitoring device 120 (e.g., when cardiovascular data from the time of admission is not yet in the EHR dataset). For example, the readmission prediction module 140 may generate one or more API calls to a medical records system cloud server 105, causing the server 105 to obtain the patient's EHR dataset from the EHR database 108B and/or obtain the cardiovascular data from the monitoring device 120. The EHR dataset may include or otherwise indicate one or more of medications, vitals, lab results, demographics, chief complaint, comorbidities, encounter diagnosis, treatments, procedures, length to previous encounter, discharge disposition, current unit, or level of care at discharge of the patient.

The MLOM 118 of the server 105 may load the machine learning model 124, and provide the client EHR dataset to the machine learning model 124. In at least some embodiments, the monitoring device 120 may generate cardiovascular data of the patient while they are admitted, which is stored in the patient's EHR dataset and/or otherwise accessible to the server 105 (e.g., transmitted from the monitoring device 120 to the server 105 via the network 110). In such embodiments, the MLOM 118 may provide the patient cardiovascular data to the machine learning model 124 as well.

The machine learning model 124 may determine a risk metric corresponding to a predicted risk of readmission of the patient for acute heart failure by analyzing the EHR dataset which may include patient cardiovascular data. The machine learning model 124 may also be trained to generate one or more outputs (e.g., metrics, predictions) associated with death of the patient (e.g., within 30 days) from acute heart failure, the readmission of the patient within 30 days for acute heart failure, and/or readmission of the patient within 60 days for acute heart failure, as previously described.

The risk metric may include a score, ranking, and/or other suitable metric. In at least some embodiments, determining the risk metric may include determining a risk classification (e.g., low risk, medium risk, or high risk of 30 day readmission for AHF), based upon the risk metric. Determining the risk classification may include associating certain values of risk metrics with stratifications of low risk, medium risk, or high risk. For example, the risk metric may have a value between 0 (no risk of 30 day readmission) and 1 (high risk of 30 day readmission, where risk metric values between 0 and 0.3 are considered low risk, risk metric values above 0.3 and below 0.6 are considered medium risk, and risk metric values between 0.6 and 1 are considered high risk, however the risk classification may include any suitable classification.

When the risk metric and/or risk classification exceeds a threshold metric and/or threshold classification respectively, the server 105 may output or otherwise provide at an output device (e.g., to the client device 115, the patient EHR dataset in the EHR database 108B) an output associated with the risk metric, such as a message, warning, notification that may indicate or otherwise include the risk metric and/or risk classification. In at least some embodiments, the output may include one or more features and/or respective values (e.g., Shapley features and respective Shapley values) associated with the EHR dataset or cardiovascular data that were most pertinent to determining the risk metric. The risk metric may be used (e.g., by the care provider) to determine a treatment of the patient (e.g., to avoid readmittance acute heart failure). The treatment may include not discharging the patient, medicine, ordering laboratory results, and/or other suitable treatments.

The computing environment 100 may include additional, fewer, and/or alternate components, and may be configured to perform additional, fewer, or alternate actions, including components/actions described herein. Although the computing environment 100 is shown in FIG. 1 as including one instance of various components such as the server 105, the network 110, the client device 115, the monitoring device 120, etc., various aspects include the computing environment 100 implementing any suitable number of any of the components shown in FIG. 1 and/or omitting any suitable ones of the components shown in FIG. 1. As one example, information described as being stored in the model training database 108A may be stored in the server memory 104, and therefore the model training database 108A may be omitted. As another example, the computing environment 100 may include hundreds of client devices 115 in communication with dozens of servers 105. Moreover, various aspects include the computing environment 100 including any suitable additional component(s) not shown in FIG. 1, such as but not limited to the exemplary components described above. Furthermore, it should be appreciated that additional and/or alternative connections between components shown in FIG. 1 may be implemented. As just one example, server 105 and the model training database 108A may be connected via a direct communication link (not shown in FIG. 1) instead of, or in addition to, via the network 110.

### Example ML Model Training

FIG. 2A depicts a combined block and logic diagram 200 for example training a machine learning model, according to some embodiments. More specifically, an ML engine 205 (e.g., the ML module 114) trains an ML model 210 (e.g., the ML models 124) using training data 215. The trained ML model 210 may be applied to, and/or receive, an input 220 and in response generate an output 225.

The ML engine 205 may include one or more hardware and/or software components to obtain, create, (re)train, operate, fine-tune, and/or store the ML model 210. A server (e.g., the server 105) or other computing device capable of model training, may obtain and/or have available one or more types of training data 215 for model creation, training, retraining and/or fine-tuning (generally referred to herein as “training”). The ML model 210 may be trained via regression, k-nearest neighbor, support vector regression, and/or random forest algorithms and/or models, although any type of applicable ML algorithm and/or training may be used, including training using one or more of supervised learning, unsupervised learning, semi-supervised learning, and/or reinforcement learning.

In at least one aspect, at least some of the training data 215 may be labeled to aid in training the ML model 210. The training data 215 may include one or more datasets, such as a training dataset (e.g., for model training), a validation dataset (e.g., for model validating/tuning), and testing datasets (e.g., to test performance on previously unseen data). The ML engine 205 may process and/or analyze the training data 215 to learn associations and/or relationships in the training data 215, and configure the ML model 210 to process the training data 215 such that when the ML model 210 receives an input 220 it has not analyzed before, it generates an appropriate outputs 225.

The training data 215 for the ML model 210 may include electronic health record dataset of a plurality of historical patients (e.g., EHR data from patient encounters at Michigan Medicine) as previously described above respective to the data stored in the EHR database 108B, cardiovascular datasets (or other data gathered by the monitoring device 120) of a plurality of historical patients, historical risk metrics of predictions of readmission (e.g., risk of 30-day readmission, 60-day readmission, readmission based on another time frame, etc.) or mortality within a time frame (e.g., death within 30 days) due to AHF of a plurality of historical patients, and/or any other suitable training data for the model 124. The ML model 210 may be trained to analyze structured and/or nonstructured training data 215, and/or may learn associations between features of the training data 215. For example, the ML model 210 may learn relationships and associations between features of the historical EHR datasets and/or cardiovascular datasets that indicate a risk of readmission within a time frame (e.g., 30-day readmission, 60-day readmission) or mortality within a time frame (e.g., death within 30 days) of a patient from AHF, associations indicating the accuracy of historical predictions (e.g., valid or invalid) of readmission within a time frame (e.g., 30-day readmission, 60-day readmission) or mortality within a time frame (e.g., death within 30 days) from AHF, and/or other suitable associations or relationships.

In some examples, the training data 215 may comprise a large source dataset that has been pre-processed to separate the source dataset into a model training dataset, a model validation dataset, and model testing dataset. The processing pipeline method may include gathering EHR datasets (e.g., of patients in a study) based on a number of criteria, then preprocessing that EHR datasets, such as pulling labs, vitals, and location information corresponding to the respective patients/study participants. That processed EHR datasets may be broken up into the training dataset, the validation dataset, and the testing dataset, for example by applying window intervals, analysis of labels, imputing missing data, applying tuning parameters, etc. The ML model 210 may be trained on the training dataset, and the validation dataset may be used during that training, for example to avoid overfitting. The testing dataset may be used to provide further evaluation of the ML model 210, by performing inference operations on the testing dataset, where that dataset is disjoint or otherwise a separate dataset from the training and validation datasets. The output of the ML model 210 in response to that testing dataset may be used to further train or tune the ML model 210. Splitting the source dataset into a model training dataset, a model validation dataset, and a model testing dataset may ensure that the ML model 210 will perform well on new, unseen data.

FIG. 2B depicts a block diagram of an example neural network 230 trained to predict patient readmission and/or mortality from AHF, according to embodiments. The architecture of the neural network 230 (e.g., the machine learning model 124, 210) may include a first dense layer 232 followed by a first dropout layer 234, followed by a second dense layer 236, followed by a second dropout layer 238, followed by a third dense layer 240. The first dense layer 232 and/or the second dense layer 236 may include a L2 regularization. The third dense layer may include a single neuron having a sigmoid activation function, and may generate the risk metric. The neural network 230 may provide benefits over other machine learning model architectures, for example, the ability to process unstructured and structured EHR data as compared to models only able to analyze structured EHR data, dropout layers and L2 regularization of the neural network 230 that beneficially reduce overfitting, and/or configuring the neural network 230 with early stopping to reduce overfitting and improve generalization.

In at least some aspects, the ML model 210 may be considered successfully trained when able to achieve one or more metrics associated with its performance when processing the training data 215. FIG. 2C depicts example metrics 250 associated with evaluating model training, according to embodiments. The metrics 250 may include a table 252 indicating area under curve (AUC or AUROC) values, and area under the precision-recall curve (AUPR or AUPRC) values associated with training the neural network 230. The AUC may be an indicator of model performance (e.g., increase in value indicating better performance) when training the model 210 to discriminate between the two classes (e.g., patient readmission or non-readmission). The AUC or AUROC value may range from 0.0-1.0, with a value of 0.5 indicating that the model performs no better than random chance and a value of 1.0 indicating that the model performs perfectly. The AUPR may be an indicator of model performance (e.g., increase in value indicating better performance) when training the model 210 to discriminate between different outcomes (identifying a specific post-patient discharge outcome, such as readmission within 30 days for AHF). The AUPR may focus on the performance of a model at detecting the minority class (e.g., detecting patient readmission), with an AUPR value of 0.5 indicating excellent performance. The metrics 250 also include a graph 254 corresponding to the AUROC value of 0.71 associated with the test dataset of the last row of the table 252.

Once trained, the ML engine 205 may load the ML model 210 at runtime to perform operations on one or more data inputs 220 to produce the desired data output 225. Namely, the model 210 receives an EHR dataset of a patient. In at least some embodiments, the model 210 may also potentially receive cardiovascular data of the patient as the input data 220 (e.g., when the cardiovascular data is included in, or available separately from, the EHR dataset), which may improve the output 225 of the model. The model 210 may analyze the input data 220 to determine a risk metric corresponding to a predicted risk of readmission and/or mortality (e.g., within 30 days from discharge, 60 days from discharge) of the patient for acute heart failure as the output 225.

One or more metrics of the trained model 210 may be compared to similar metrics of other models to determine whether the model 210 outperforms other, similar models (e.g., models with different tuning, different types of models, etc.). FIG. 2D depicts an example table 260 indicating an AUROC performance metric for evaluating similar predictive models, according to embodiments. As shown, the model 210 may perform as well or better than all other comparable models that address AHF and utilize also statistically robust methods. For example, the model 210 may have an AUROC value of 0.71, indicating the model 210 may perform better than the Sudhakar et al. model (0.61), Fleming et al. model (0.66), Wang et al. model (LACE index) (0.59), Zai et al. telemonitoring-based readmission model (0.21), Zai et al. psychosocial model (0.67), Au administrative data-based models (range from 0.57-0.61), Watson psychosocial readmission model (0.67), Di Tano et al. model (0.695), and the Huynh et al. non-clinical model (0.66). Other models may also be less statistically robust and trained on a limited number of patients and/or include bias, which may artificially inflate the AUROC value.

The server, the ML engine 205, and/or other suitable device may update the training data 215 at one or more times. The ML model 210 may be retrained based upon the updated training data 215, the retrained/updated ML model 210 may be stored in memory (e.g., the memory 104) and/or subsequently executed. Model retraining may cause the output 225 of the ML model 210 to improve over time. For example, the training data may be updated with new EHR datasets. The ML engine 205 may train the model 210 using the updated training data, and in the process make new associations between the features of the updated EHR dataset, such that the risk metrics generated as the output 225 are more accurate than before model retraining.

### Exemplary Clinical Workflow

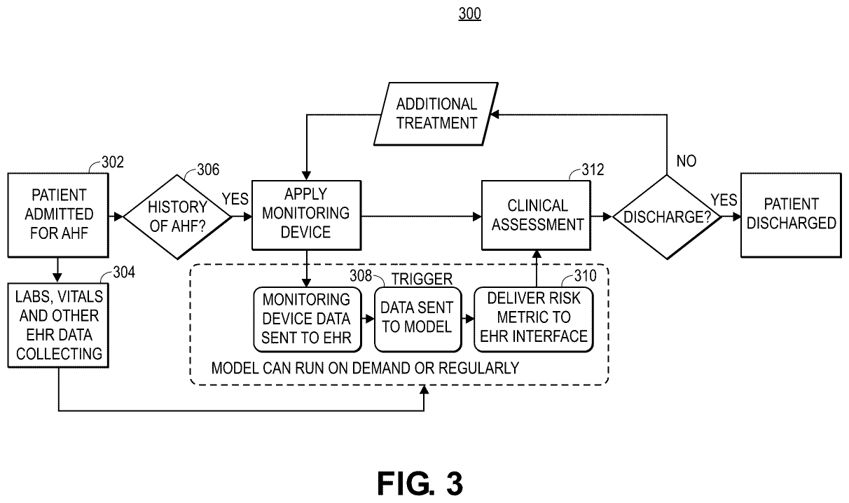

FIG. 3 depicts an example clinical workflow 300 for predicting readmission of a patient for acute heart failure using machine learning, according to embodiments. The clinical workflow 300 may be performed via the computing environment 100, for example a cloud-computing EHR system. At block 302, a patient is admitted to a care facility for AHF, and at block 304 a collection of EHR data of the patient may begin while they are admitted, such as labs, vitals, or other suitable EHR data. At block 306, the care facility may determine whether to monitor the patient via a monitoring device (e.g., the monitoring device 120), for example based upon whether the patient has a history of AHF.

One or more triggers may cause a model (e.g., the model 124, 210) to receive EHR data of the patient at block 308. As shown, in some examples, monitoring device data, such as real-time monitoring device data, may be sent to an EHR system for inclusion in the EHR data. A trigger may include a request of a caregiver at the time of patient discharge, the discovery of a patient (e.g., via a routine running in the background of the EHR record system) admitted for AHF, a patient admitted for AHF that receives intravenous diuretics (e.g., as indicated by ICD codes in the EHR), a discharge event (e.g., electronic discharge request, EHR information indicating suggested discharge, etc.) of a patient admitted with AHF, and/or any other suitable trigger. At block 310, the model may analyze the EHR data of the patient collected while the patient is admitted for AHF (i.e., with monitoring device data has been sent to be included in the EHR data) to generate the risk metric associated with 30-day readmission, 60-day readmission, and/or mortality due to AHF after discharge. In some examples, when the monitoring device data is sent for inclusion in the EHR data, pre-processing may be performed on the multimodal, asynchronous, and sometimes noisy clinical data streams, such as existing EHR data in combination with real-time or near real-time physiological data generated by distributed monitoring devices. These and other pre-processing techniques herein may be used in various examples, including various examples where machine learning architectures are trained through transforming heterogeneous medical data sources into a unified computational representation suitable for machine learning. Returning to FIG. 3, as shown, block 310 may include providing risk metric and/or other associated information (e.g., features and their values most relevant to the risk metric) at an EHR interface (e.g., the user interface 138).

At block 312, the care provider of the patient may perform a clinical assessment based upon the risk metric, improving the quality of care of the patient and reducing the risk of penalization for patient admission. The clinical assessment or otherwise treatment decision may include whether to continue with a discharge the patient (e.g., based upon low risk of readmission or mortality from AHF), provide additional treatment to the patient (e.g., to mitigate a predicted likelihood of readmission or mortality from AHF), and/or other suitable course of action.

### Example Method for Predicting Readmission of a Patient Within 30 Days for Acute Heart Failure Using Machine Learning

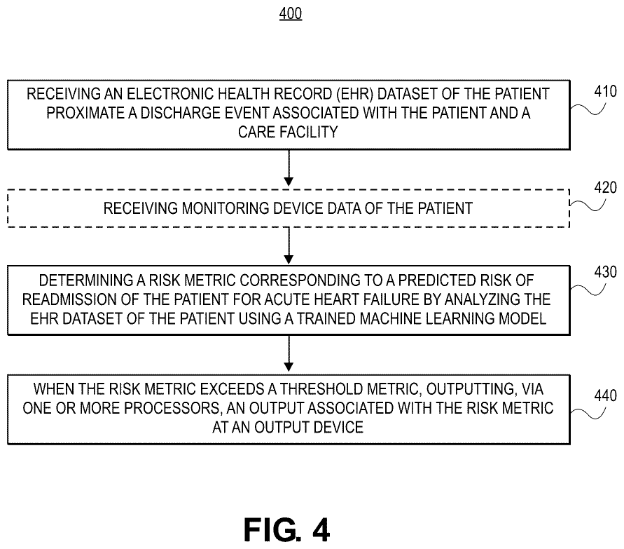

FIG. 4 depicts a flow diagram of an example computer-implemented method 400 for predicting readmission of a patient for acute heart failure using machine learning, according to embodiments. One or more steps of the method 400 may be implemented as a set of instructions stored on a computer-readable memory and executable via one or more local or remote processors (e.g., the processor 102, 132) and/or other electronic or electrical devices and/or components, which may be in wired or wireless communication with one another.

The method 400 may include receiving an electronic health record (EHR) dataset of the patient proximate a discharge event associated with the patient and a care facility (block 410). That medical EHR dataset may include data from one or more monitoring devices, including historical monitoring device data from previous monitoring of the subject. In some examples, the method 400 may include receiving monitoring device data of the patient, wherein the monitoring device data is captured in real time from the subject. In some examples, the method 400 may receive both an EHR dataset (block 410) and monitoring data from one or more monitoring devices (block 420), such as cardiovascular data. In at least some embodiments, the method 400 may include obtaining cardiovascular data from at least one monitoring device monitoring the patient during admission (e.g., monitoring device data), wherein the risk metric is further based upon the trained machine learning model analyzing the cardiovascular data. The EHR dataset may indicate the patient, while admitted to the care facility, is diagnosed with acute heart failure and receives a medication associated with acute heart failure. The EHR dataset may indicate one or more of medications, vitals, lab results, demographics, chief complaint, comorbidities, encounter diagnosis, treatments, procedures, length to previous encounter, discharge disposition, current unit, or level of care at discharge. The medication associated with acute heart failure may be an intravenous diuretic.

The method 400 may include determining a risk metric corresponding to a predicted risk of readmission of the patient for acute heart failure by analyzing the EHR dataset of the patient using a trained machine learning model (e.g., the machine learning model 124, 210) (block 430).

The method may include outputting an output associated with the risk metric at an output device when the risk metric exceeds a threshold metric (block 440). The output associated with the risk metric may be used to determine a treatment of the patient to avoid readmittance for acute heart failure. The output associated with the risk metric may include one or more features and one or more respective values (e.g., features and/or values of importance to the risk metric, such as Shapley features and/or values).

In at least some embodiments, trained machine learning model includes a neural network (e.g., the neural network 230). The neural network may be trained using a binary cross-entropy loss function. The neural network may include a first dense layer (e.g., the first dense layer 232), a first dropout layer (e.g., the first dropout layer 234), a second dense layer (e.g., the second dense layer 236), a second dropout layer (e.g., the second dropout layer 238), and a third dense layer (e.g., the third dense layer 240). One or more of the first dense layer or the second dense layer may include L2 regularization. The third dense layer may include a single neuron having a sigmoid activation function.

In at least some embodiments, the trained machine learning model may be trained to predict readmission of the patient with 30 days from acute heart failure and/or readmission of the patient within 60 days for acute heart failure, and further trained to predict death of the patient from acute heart failure.

In at least some embodiments, determining the risk metric may include determining a risk classification of low risk, medium risk, or high risk, based upon the risk metric, wherein outputting the output associated with the risk metric is based upon the risk classification exceeding a threshold classification.

In at least some embodiments, the present techniques include tuning ML models to learn updates to threshold levels, such as thresholds for risk classification. Tuning thresholds can thus tune the predictions that is systematic and allows for additional factors outside of the EHR data, etc. to be used, additional factors such as electronic guidelines from administrative computing systems. That is, the exact same training, validation, and testing datasets can be used on two different systems to generate the same ML models, but introducing additional data may cause one system to have a tuned set of thresholds that make that that system different than the other system. In some embodiments, the additional factors may be user-determined factors.

It should be understood that not all blocks of the exemplary flow diagram of FIG. 4 are required to be performed.

It should be understood that the present techniques may be integrated into other machine learning (or other trained services) classifications, to provide multidimensional classifications of predicted patient healthcare outcomes.

### ADDITIONAL CONSIDERATIONS

Although the text herein sets forth a detailed description of numerous different embodiments, it should be understood that the legal scope of the invention is defined by the words of the claims set forth at the end of this patent. The detailed description is to be construed as exemplary only and does not describe every possible embodiment, as describing every possible embodiment would be impractical, if not impossible. One could implement numerous alternate embodiments, using either current technology or technology developed after the filing date of this patent, which would still fall within the scope of the claims.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based upon any statement made in any section of this patent (other than the language of the claims). To the extent that any term recited in the claims at the end of this disclosure is referred to in this disclosure in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based upon the application of 35 U.S.C. 112(f).

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a central processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a processor configured using software, the processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

What is claimed:

1. A computer-implemented method for predicting readmission of a patient for acute heart failure using machine learning, the computer-implemented method comprising:
receiving, via one or more processors, an electronic health record (EHR) dataset of the patient proximate a discharge event associated with the patient and a care facility, wherein:
the EHR dataset indicates the patient, while admitted to the care facility, is diagnosed with acute heart failure and receives a medication associated with acute heart failure;
determining, via the one or more processors, a risk metric corresponding to a predicted risk of readmission of the patient for acute heart failure by analyzing the EHR dataset of the patient using a trained machine learning model; and
when the risk metric exceeds a threshold metric, outputting, via one or more processors, an output associated with the risk metric at an output device.

2. The computer-implemented method of claim 1, further comprising:
obtaining, via the one or more processors, cardiovascular data from at least one monitoring device monitoring the patient during admission, wherein the risk metric is further based upon the trained machine learning model analyzing the cardiovascular data.

3. The computer-implemented method of claim 1, wherein the trained machine learning model includes a neural network.

4. The computer-implemented method of claim 3, wherein the neural network is trained using a binary cross-entropy loss function.

5. The computer-implemented method of claim 3, wherein the neural network includes a first dense layer, a first dropout layer, a second dense layer, a second dropout layer, and a third dense layer.

6. The computer-implemented method of claim 5, wherein one or more of the first dense layer includes L2 regularization or the second dense layer includes L2 regularization.

7. The computer-implemented method of claim 5, wherein the third dense layer includes a single neuron having a sigmoid activation function.

8. The computer-implemented method of claim 1, wherein the trained machine learning model is trained to predict readmission of the patient within 30 days for the acute heart failure and/or readmission of the patient within 60 days for the acute heart failure, and is further trained to predict death of the patient from acute heart failure.

9. The computer-implemented method of claim 1, wherein determining the risk metric comprises:
determining a risk classification of low risk, medium risk, or high risk, based upon the risk metric, wherein outputting the output associated with the risk metric is based upon the risk classification exceeding a threshold classification.

10. The computer-implemented method of claim 1, wherein the EHR dataset indicates one or more of medications, vitals, lab results, demographics, chief complaint, comorbidities, encounter diagnosis, treatments, procedures, length to previous encounter, discharge disposition, current unit, or level of care at discharge.

11. The computer-implemented method of claim 1, wherein the output associated with the risk metric is used to determine a treatment of the patient to avoid readmittance for acute heart failure.

12. The computer-implemented method of claim 1, wherein the medication associated with acute heart failure is an intravenous diuretic.

13. A system for predicting readmission of a patient for acute heart failure using machine learning, comprising:
one or more processors, and
a memory including computer executable instructions that, when executed by the one or more processors, cause the system to:
receive an electronic health record (EHR) dataset of the patient proximate a discharge event associated with the patient and a care facility, wherein:
the EHR dataset indicates the patient, while admitted to the care facility, is diagnosed with acute heart failure and receives a medication associated with acute heart failure;
determine a risk metric corresponding to a predicted risk of readmission of the patient for acute heart failure by analyzing the EHR dataset of the patient using a trained machine learning model; and
when the risk metric exceeds a threshold metric, output an output associated with the risk metric at an output device.

14. The system of claim 13, wherein further comprising instructions that, when executed by the one or more processors, cause the system to:
obtain cardiovascular data from at least one monitoring device monitoring the patient during admission, wherein the risk metric is further based upon the trained machine learning model analyzing the cardiovascular data.

15. The system of claim 13, wherein the trained machine learning model includes a neural network.

16. The system of claim 15, wherein the neural network is trained using a binary cross-entropy loss function.

17. The system of claim 15, wherein the neural network includes a first dense layer, a first dropout layer, a second dense layer, a second dropout layer, and a third dense layer.

18. The system of claim 17, wherein one or more of:
the first dense layer includes L2 regularization;
the second dense layer includes L2 regularization; or
the third dense layer includes a single neuron having a sigmoid activation function.

19. The system of claim 13, wherein the trained machine learning model is trained to predict readmission of the patient within 30 days for the acute heart failure and/or readmission of the patient within 60 days for the acute heart failure, and is further trained to predict death of the patient from acute heart failure.

20. A non-transitory computer readable medium storing instructions that, when executed by one or more processors, causes the one or more processors to at least:
receive an electronic health record (EHR) dataset of a patient proximate a discharge event associated with the patient and a care facility, wherein:
the EHR dataset indicates the patient, while admitted to the care facility, is diagnosed with acute heart failure and receives a medication associated with acute heart failure;
determine a risk metric corresponding to a predicted risk of readmission of the patient for acute heart failure by analyzing the EHR dataset of the patient using a trained machine learning model; and
when the risk metric exceeds a threshold metric, output an output associated with the risk metric at an output device.
